(19) United States
(12) Patent Application Publication
(10) Pub. No.: US 2026/0278695 A1
(43) Pub. Date: Sep. 17, 2026

(54) Agentic Generative AI for Predictive Modelling of Reimbursement Decisions and Health Technology Assessment

(71) Applicant: Loon Inc., Ottawa (CA)

(72) Inventors: Mara Uzun, Cantley (CA); Ghayath Janoudi, Cantley (CA)

(21) Appl. No.: 19/079,618

(22) Filed: Mar. 14, 2025

(51) Int. Cl. G06Q 40/12; G06Q 10/067; G16H 10/60

(52) U.S. Cl. CPC G06Q 40/12; G06Q 10/067; G16H 10/60

(57) ABSTRACT

A computer-implemented method for predictive modelling of reimbursement decisions for a health technology includes receiving input data that defines a health technology and parameters of a target Health Technology Assessment (HTA) scenario. The input data is analyzed by a generative artificial intelligence (AI) model trained on historical HTA decision data to produce a structured scenario profile. Contextual data relevant to the structured scenario profile is retrieved from multiple databases, including a historical HTA decision database, a clinical evidence database, and a health economic evidence database. A deliberation process is simulated using multiple AI agents, each representing a distinct HTA expert perspective, engaging in iterative interactions to determine a simulated reimbursement decision outcome. The method generates recommendations for modifying an HTA submission dossier to increase the likelihood of an affirmative reimbursement decision. The simulated reimbursement decision outcome and recommendations are provided to the user device.

### FIELD OF THE INVENTION

The field of the invention is healthcare technology and, in particular, a system and method for predictive modelling of reimbursement decisions and health technology assessments using agentic generative artificial intelligence.

### BACKGROUND OF THE INVENTION

The following description includes information that may be useful in understanding the present invention. It is not an admission that any of the information provided herein is prior art or relevant to the presently claimed invention, or that any publication specifically or implicitly referenced is prior art.

In healthcare, obtaining reimbursement approval for new pharmaceuticals or medical technologies requires rigourous Health Technology Assessment (HTA) processes by payer and regulatory bodies. Expert committees evaluate clinical effectiveness, cost-effectiveness, and other factors before deciding whether to recommend a product for coverage. Predicting these reimbursement decisions is challenging due to the complex, multi-factorial deliberations involved. Traditionally, pharmaceutical companies rely on health economists and policy experts to anticipate outcomes, using static models or past experience. These conventional approaches often cannot capture the dynamic and deliberative nature of committee decision-making, leading to uncertainty in market access planning.

Moreover, reimbursement decision outcomes depend on continually evolving data new clinical trial results, real-world evidence, changing treatment guidelines, and shifting budget impact considerations. Existing prediction tools, if any, may not integrate real-time data or multi-perspective analysis effectively. They tend to be rule-based or use single-dimensional criteria (e.g., cost-effectiveness thresholds) and thus struggle with specificity and accuracy. As a result, companies face substantial risk in HTA submissions, which can lead to less-than-optimal payer negotiation strategies or even unexpected negative recommendations that delay patient access to innovations.

Thus, there is a need for an improved system that can simulate the expert committee deliberation process and provide predictions of HTA outcomes and to mitigate some of the errors inherent in current approaches. Methods and systems that provide more reliable, adaptive, and comprehensive analysis of clinical and economic data, and improved market access strategies and patient access to innovative therapies are needed.

### BRIEF DESCRIPTION OF DRAWINGS

Various objects, features, aspects, and advantages of the inventive subject matter will become more apparent from the following detailed description of preferred embodiments, along with the accompanying drawing figures in which like numerals represent like components.

FIG. 1 is a block diagram of a system architecture for an HTA decision modelling system in accordance with an example of the present specification.

FIG. 2 is a diagram illustrating a conceptual overview of the HTA decision modelling process, in accordance with an example of the present specification.

FIG. 3 is a flow diagram for an operational process of the HTA decision modelling system of FIG. 1.

FIG. 4 to FIG. 11 are screenshots of a client application in accordance with an example of the present specification.

### DETAILED DESCRIPTION OF THE INVENTION

This detailed description provides an explanation of the embodiments of the present specification. The present specification encompasses a variety of systems, methods, and non-transitory computer-readable media.

A method for predictive modelling of reimbursement decisions for a health technology, implemented by a computing system, includes: receiving, from an electronic user device, input data comprising a description of a health technology and parameters defining a target HTA scenario; analyzing the input data using at least one generative artificial intelligence (AI) model trained on historical HTA decision data to generate a structured scenario profile; retrieving contextual data relevant to the structured scenario profile from a plurality of databases comprising at least a historical HTA decision database, a clinical evidence database, and a health economic evidence database; simulating a deliberation process for evaluating the health technology in the target HTA scenario using a plurality of AI agents, each configured to represent a distinct expert perspective relevant to HTA deliberations, and wherein the AI agents interact iteratively by exchanging evaluations to determine a simulated reimbursement decision outcome; generating the simulated reimbursement decision outcome based on the deliberation among the AI agents; generating one or more recommendations for modifying an HTA submission dossier associated with the health technology to increase a likelihood of receiving an affirmative reimbursement decision outcome; and providing, to the electronic user device, the simulated reimbursement decision outcome and the one or more recommendations. Advantageously, this method enhances predictive accuracy and supports preparation of HTA submissions using artificial intelligence techniques.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of features used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed considering the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

A computing system may include one or more processors, memory, and storage devices configured with software and/or firmware to implement the specified functionalities. The hardware is designed to support the computational demands of agentic and generative AI methods including but not limited to large language models, Bayesian analysis, and reinforcement learning techniques.

The system may process healthcare data and reimbursement-related data through a series of computational steps, including data collection, cleaning, normalization, and analysis. Machine learning algorithms implemented on the system can analyze large datasets to identify patterns, simulate expert deliberations, generate predictions or recommendations based on trained AI models.

Training of machine learning models involves feeding historical datasets into the system, where data is used to adjust model parameters. The training process can utilize various learning algorithms, including supervised learning, unsupervised learning, or reinforcement learning techniques, depending on the nature of the reimbursement prediction task being addressed. Fine-tuning of generative AI models, such as pre-trained large language models, can also be performed to adapt models specifically to healthcare reimbursement and HTA scenarios.

The system includes a user interface that allows users to interact with its various functionalities, providing inputs, configuring settings, and receiving outputs. This interface can be provided through web-based, mobile, or desktop applications designed to facilitate user interaction with the predictive modelling processes and to display results in an understandable and actionable manner.

The system can be implemented on cloud-based infrastructure, allowing scalable computing resources and storage capacity suitable for large-scale applications. Networking technologies enable the system to access distributed healthcare data sources, integrate with external databases and other systems, and provide services to remote users over networks such as the Internet.

A system of one or more computers can be configured to perform particular operations or actions by virtue of having software, firmware, hardware, or a combination thereof installed on the system that, in operation, cause or causes the system to perform the actions. One or more computer programs can be configured to perform particular operations or actions by including instructions that, when executed by data processing apparatus, cause the apparatus to perform the described operations or actions.

A Health Technology Assessment (HTA) is a systematic evaluation of medical technologies, treatments, pharmaceuticals, or healthcare interventions designed to inform reimbursement and coverage decisions made by payer organizations and regulatory bodies. HTA processes typically assess clinical effectiveness, cost-effectiveness, patient safety, ethical implications, and societal impacts associated with the health technology under consideration. Typically, these assessments are conducted by expert committees composed of healthcare professionals, clinicians, health economists, patient representatives, policy experts, epidemiologists, and biostatisticians, each contributing specialized perspectives for informed decision-making. The multidisciplinary nature of HTA committees is equipped to review both clinical and economic evidence, ultimately influencing whether a technology is recommended for reimbursement, restricted use, or exclusion from healthcare coverage.

An HTA recommendation typically specifies whether a health technology should be reimbursed by a healthcare system and under what conditions such reimbursement should occur. Recommendations may include unconditional approval, approval with specific clinical or economic conditions, restricted coverage limited to particular patient populations or disease subgroups, or non-approval due to insufficient clinical evidence or unfavourable economic outcomes. Conditions accompanying positive recommendations often involve measures such as patient eligibility criteria, requirements for additional clinical evidence generation, mandated price reductions or negotiated discounts, implementation of risk-sharing agreements, or outcome-based reimbursement frameworks designed to manage budget impact and clinical uncertainty. Such recommendations serve as relevant guidance for payers, healthcare providers, and technology sponsors regarding patient access, reimbursement strategies, and future research or data-generation requirements.

The HTA process typically requires extensive preparation, including the development of comprehensive clinical dossiers, health economic analyses, and systematic reviews, which can take one to two years or more. Once submitted, expert committees may deliberate for several months before rendering a reimbursement decision. Appeals or revisions following initial recommendations may extend the process by additional years, delaying patient access to innovative treatments and technologies. Given the considerable time, resources, and financial investment associated with each HTA submission, accurate predictive modelling of HTA outcomes becomes important. Forecasting enables stakeholders to proactively refine submissions, address potential committee concerns, mitigate risks associated with unfavourable decisions, and enhance the likelihood of timely and successful reimbursement outcomes, ultimately improving patient access to medical innovations.

An affirmative or favourable HTA decision typically involves a recommendation by the expert committee that the health technology be reimbursed or covered by healthcare payers, allowing patient access under clearly defined conditions. Such positive outcomes can range from full and unconditional reimbursement, indicating strong clinical and economic justification, to conditional reimbursement subject to specific constraints or requirements.

A payer, in the context of healthcare reimbursement, refers to an entity responsible for funding or reimbursing the cost of medical treatments, pharmaceuticals, and healthcare services. Payers can include government health agencies, public healthcare systems, private insurance companies, and pharmacy benefit managers (PBMs).

An HTA submission dossier is a comprehensive, structured document prepared by the sponsor of a health technology—such as a pharmaceutical manufacturer or medical device company—to support reimbursement decisions made by payer or regulatory bodies. The dossier typically compiles extensive clinical evidence, including trial results, real-world evidence, patient-reported outcomes, health economic analyses, and budget impact assessments. In practice, the dossier submitted to a particular jurisdiction is often adapted or derived from a global value dossier or from materials previously prepared for earlier submissions in other regions or countries, reflecting local epidemiological contexts, healthcare standards, clinical practices, and economic constraints specific to that jurisdiction. The clinical evidence included in such dossiers generally originates from phased clinical trials: Phase 1 studies assess safety and dosage in healthy volunteers or small patient groups; Phase 2 studies explore preliminary efficacy and further safety in patients diagnosed with the condition; and Phase 3 studies involve larger, randomized clinical trials intended to establish definitive evidence of efficacy and safety for regulatory approval and HTA submissions.

Real-world evidence (RWE) refers to clinical and economic data derived from sources outside controlled clinical trial settings, including observational studies, patient registries, electronic health records, insurance claims databases, and other routinely collected health information. RWE provides insights into the effectiveness, safety, adherence, and economic impacts of health technologies under actual clinical practice conditions. Increasingly, payers and HTA bodies rely on RWE to inform reimbursement decisions, address uncertainties associated with controlled clinical trials, and support post-marketing surveillance and ongoing assessments of real-world outcomes.

A foundational large language model (LLM) refers to a generative artificial intelligence model trained on vast amounts of general textual data to learn linguistic patterns, semantic relationships, and reasoning capabilities. Such foundational models, prior to fine-tuning, possess broad and generalized knowledge but are not inherently specialized or optimized for specific tasks or domains. Un-fine-tuned foundational LLMs generate outputs based on probabilistic predictions derived from the extensive but general training corpus, and their outputs may vary in accuracy and relevance when applied directly to specialized contexts, such as healthcare reimbursement decisions. Generative AI encompasses computational models capable of generating novel and contextually relevant outputs-such as textual summaries, analytical insights, strategic recommendations, or predictive outcomes-by synthesizing patterns learned from large-scale training data. In the HTA context, generative AI can produce analyses resembling those of human experts by interpreting and integrating clinical evidence, health economic data, reimbursement precedents, and payer policies to simulate realistic deliberative discussions and forecast reimbursement decisions.

Fine-tuning of foundational large language models involves adapting the general capabilities of these pre-trained models to specific domains or expert tasks by providing targeted, high-quality, domain-specific training data. In the context of predictive modelling for HTAs, fine-tuning transforms general-purpose LLMs into specialized AI agents capable of emulating the expert perspectives required for accurate reimbursement decision simulations. This domain-specific training process incorporates curated datasets, including historical HTA committee transcripts, published reimbursement decisions, detailed clinical and economic evidence, and documented expert deliberations relevant to distinct areas of expertise.

According to one example of the present specification, each AI agent used within the predictive modelling system is fine-tuned independently to develop specialized expertise reflective of an HTA committee member's perspective. For instance, a clinical evidence agent is fine-tuned on clinical efficacy and safety data from regulatory dossiers, pivotal clinical trial results, systematic reviews, and clinical practice guidelines. A health economics agent is fine-tuned using cost-effectiveness analyses, budget impact models, economic evaluations, and payer decision documents. A policy expert agent is fine-tuned on datasets comprising HTA frameworks, reimbursement guidelines, payer policies, committee deliberation records, and ethical considerations documented in HTA assessments.

Through such targeted fine-tuning, each AI agent acquires the ability to analyze and reason effectively within its specialized domain, replicating expert deliberations and judgements encountered in real-world HTA processes. This fine-tuning greatly enhances the predictive accuracy, specificity, and relevance of simulated reimbursement outcomes generated by the predictive modelling system, thereby providing actionable insights and meaningful recommendations tailored to optimize HTA submissions and strategic reimbursement planning.

According to one example of the present specification, fine-tuning a foundational LLM typically involves initializing the model with pre-trained weights derived from general training corpora, then iteratively updating these weights. For example, OpenAI™ enables fine-tuning through accessible Application Programming Interfaces (APIs), where users submit their domain-specific training datasets to fine-tune a pre-trained model remotely on OpenAI's infrastructure, simplifying deployment and management. Alternatively, organizations may choose to self-host and fine-tune open-source models, such as Meta's LLAMA™ series, on their own cloud or local infrastructure, providing greater flexibility, data privacy, and control over model deployment and inference optimization.

Agentic AI, as used herein, refers to specialized generative AI models configured as discrete, autonomous agents, each trained to emulate a distinct expert role typically represented on HTA committees. These agents independently assess specific domains of evidence—such as clinical trial outcomes, cost-effectiveness data, patient perspectives, or regulatory frameworks—and subsequently engage in structured interactions with other agents to simulate the dynamics of expert committee deliberations. According to examples of the present specification, agentic AI denotes AI modules designed and fine-tuned to replicate targeted expert judgments within iterative and structured deliberations.

As used in this specification, iterative deliberation (also referred to as discussion cycles) describes the structured, cyclical exchange of evaluations and arguments between multiple AI agents. During each iterative cycle, agents generate outputs reflecting their specialized expert perspectives, considering both their independent analysis of evidence and information received from other agents. Subsequent deliberation cycles incorporate these updated perspectives, allowing agents to refine their evaluations iteratively. Iterations continue until predefined convergence criteria-such as reaching a stable consensus, minimizing variance in predictions, or achieving a threshold of alignment-are satisfied, resulting in robust and thoroughly vetted reimbursement decision predictions.

Dynamic updating refers to the capability of the predictive modelling system to incorporate emerging or real-time data into ongoing deliberations or simulations, ensuring that predictions remain reflective of the current clinical and policy landscape. This process can include integrating newly published clinical trial results, updates in treatment guidelines, changes in regulatory or payer policies, or fresh insights from contemporaneous HTA decisions or real-world evidence sources. Dynamic updating enhances the adaptability and timeliness of reimbursement predictions, allowing stakeholders to continuously align their submissions and strategies with the evolving HTA environment.

The confidence score generated by the predictive modelling system quantifies the likelihood, robustness, or reliability of a simulated reimbursement decision outcome. It is typically calculated by evaluating the degree of agreement or consensus achieved among multiple AI agents during iterative deliberations, the consistency of predictions over multiple simulation runs, and historical accuracy benchmarks validated against previously observed reimbursement decisions. Such confidence scores enable users to assess predictive certainty, manage submission risks, and strategically prioritize actions based on the predicted probability of achieving affirmative reimbursement decisions.

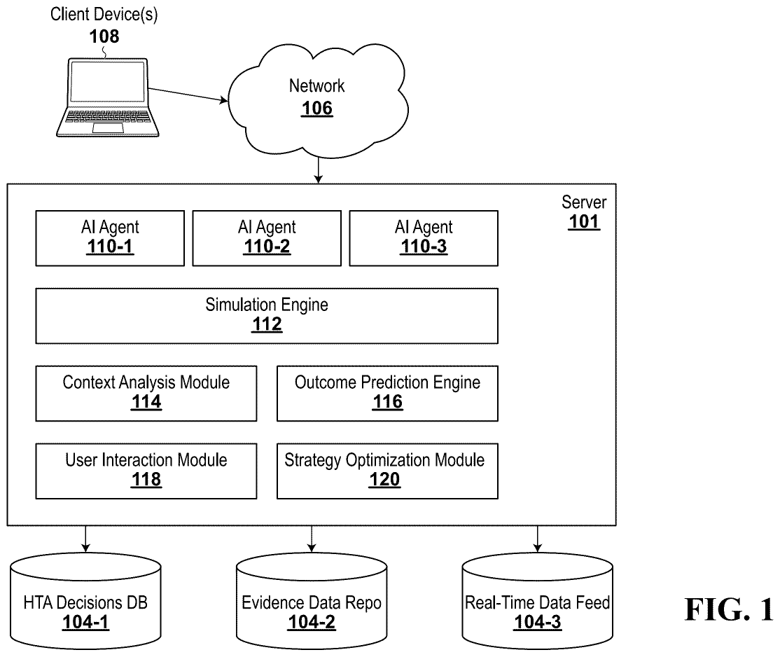

Turning to the drawings, FIG. 1 depicts a system architecture diagram for a predictive modelling system 100, according to an example of the present specification. The system 100 includes a server 101, which hosts several functional modules collectively designated as 102. These modules include a plurality of AI agent modules 110-1, 110-2, 110-3 through 110-n, a simulation engine 112, a context analysis module 114, an outcome prediction engine 116, a user interaction module 118, and a strategy optimization module 120. Each module contributes to the predictive modelling of reimbursement decisions, as detailed further below.

The server 101 communicates with multiple data storage repositories, collectively shown as databases 104, which include at least a historical HTA decision database 104-1, a clinical evidence database 104-2, and a health economic evidence database 104-3. These databases store structured and unstructured contextual data that informs the analyses performed by the AI agents 110.

Connected via network 106, the server 101 interfaces with user electronic devices 108, allowing input of scenario data, user interaction, and retrieval of predictive outputs and recommendations. Network 106 may include internet or intranet infrastructure, facilitating secure and efficient data transfer between components.

The AI agent modules 110-1 through 110-n are specialized generative AI models, each fine-tuned to represent distinct expert perspectives relevant to HTA, such as clinical evidence evaluation, health economics analysis, patient perspectives, and policy considerations. These agents analyze contextual data and independently produce evaluations relevant to their assigned expertise.

The simulation engine module 112 orchestrates structured, iterative deliberations between the multiple AI agents. It manages cycles of interaction, during which AI agents exchange their evaluations, refine their positions, and iteratively move towards consensus or convergence regarding the reimbursement decision outcome.

The context analysis module 114 dynamically retrieves and updates contextual data used during simulations. Module 114 ensures the deliberation process incorporates the latest clinical trial outcomes, real-world evidence, recent reimbursement decisions, and regulatory or clinical guideline changes, enabling the predictive modelling system to remain current and accurate.

The outcome prediction engine 116 synthesizes the evaluations and deliberations of the AI agents to generate a simulated reimbursement decision outcome. Module 116 assesses agent interactions and applies consensus algorithms or weighted decision-making methods to arrive at a coherent and reliable forecasted reimbursement decision.

The user interaction module 118 manages interactions between the predictive modelling system 100 and user electronic devices 108, handling user inputs, presenting predictive outcomes, recommendations, and confidence scores, and facilitating user interactions to refine scenario parameters and optimize submission dossiers.

The strategy optimization module 120 generates tailored recommendations to modify HTA submission dossiers, informed by deliberations of the AI agents. Module 120 identifies strategic adjustments—such as pricing modifications, targeted evidence generation, or risk-sharing agreements—that can positively influence reimbursement decisions.

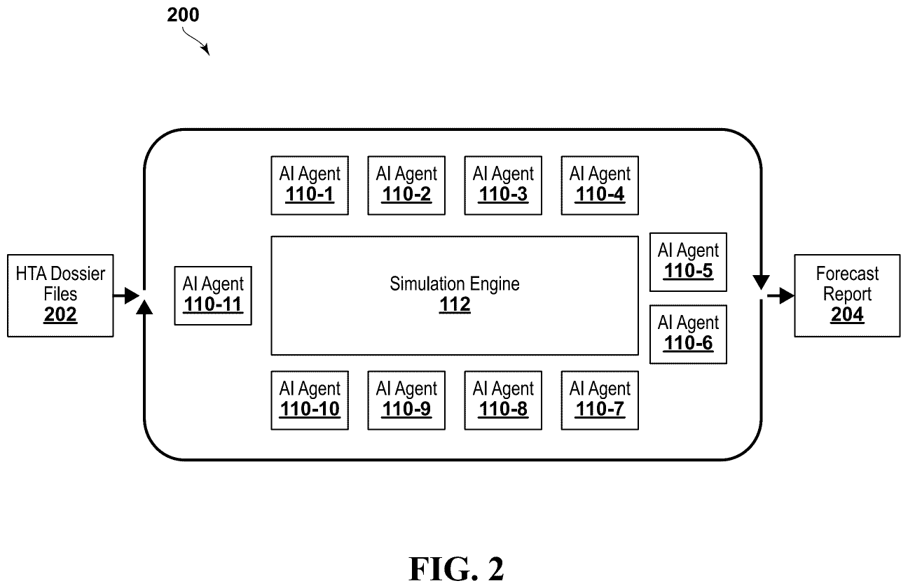

FIG. 2 illustrates a conceptual overview diagram of the HTA predictive modelling workflow in accordance with an example of the present specification. The diagram illustrates HTA dossier files 202 entering system 100, specifically directed into the simulation engine 112 that orchestrates iterative deliberations between the plurality of AI agents 110-1 through 110-n. Following deliberations and consensus, the simulation engine 112 outputs a forecast report file 204, which provides the predicted reimbursement decision outcome, associated confidence score, and tailored recommendations for improving dossier submissions.

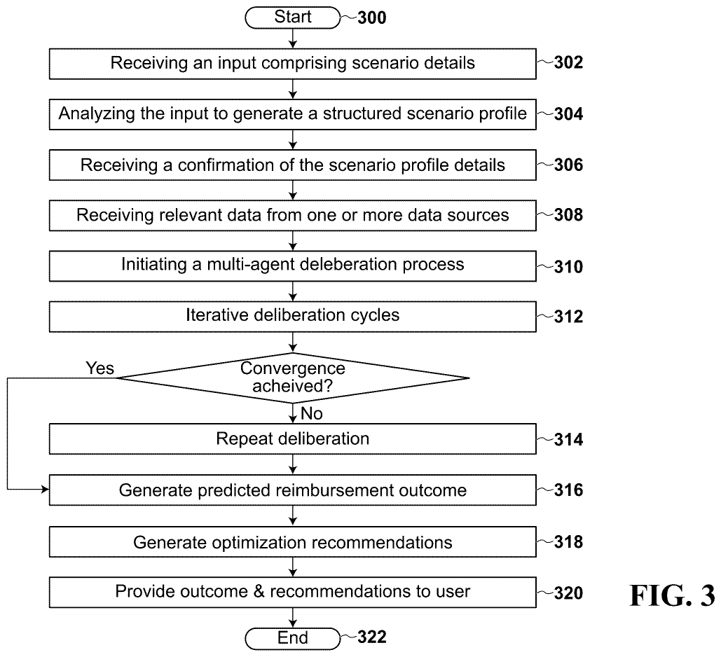

FIG. 3 is a detailed flow diagram depicting the operational process of predictive reimbursement decision modelling according to an example of the present specification. The process commences at 300. At 302, the system receives scenario input data from the user electronic device 108, containing a structured description of a health technology and associated parameters defining the target HTA context. At 304, the system generates a structured scenario profile using at least one fine-tuned generative AI model trained on historical HTA data. At 306, contextual data relevant to the structured scenario profile is retrieved from databases 104-1, 104-2, and 104-3. At 308, the system instantiates multiple AI agents 110-1 through 110-n, each assigned to evaluate evidence from distinct expert perspectives. At 310, the iterative deliberation cycle begins, where AI agents independently produce and exchange evaluations, interacting to refine their analyses. At 312, the system dynamically updates contextual data to reflect real-time changes in evidence or guidelines. At 314, a decision evaluates whether consensus or a convergence criterion is achieved. If not, iterative deliberation 310 repeats. Once consensus is reached, the simulation engine 112 produces a simulated reimbursement decision outcome at 316. At 318, the strategy optimization module 120 generates recommendations to optimize dossier submissions. At 320, the predicted reimbursement decision outcome and strategic recommendations are provided to the user electronic device 108. The process concludes at 322.

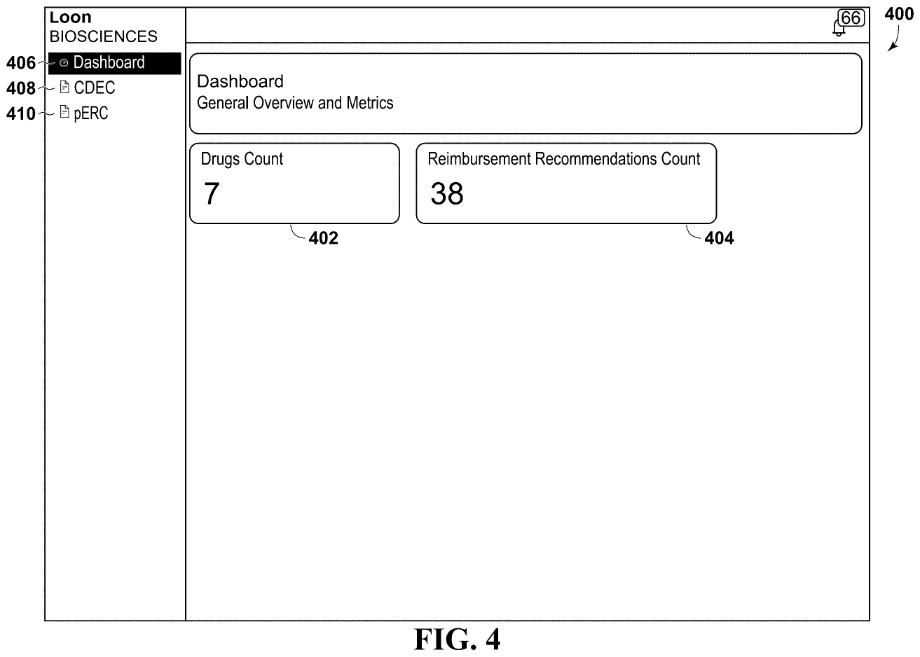

FIG. 4 illustrates a screenshot 400 of a user interface dashboard displaying general metrics and navigational buttons used within the system, according to an example of the present specification. Metric displays include “Drugs Count” 402 and “Reimbursement Recommendations Count” 404. Navigational buttons facilitate access to distinct functional areas, specifically “Dashboard” 406, “CDEC” 408, and “pERC” 410, guiding user interaction through predictive modelling scenarios. CDEC refers to the Canadian Drug Expert Committee, the primary expert advisory body of CADTH (Canadian Agency for Drugs and Technologies in Health), responsible for making recommendations regarding the reimbursement of non-oncology pharmaceuticals in Canada. pERC refers to pan-Canadian Oncology Drug Review Expert Review Committee, responsible specifically for oncology drug assessments.

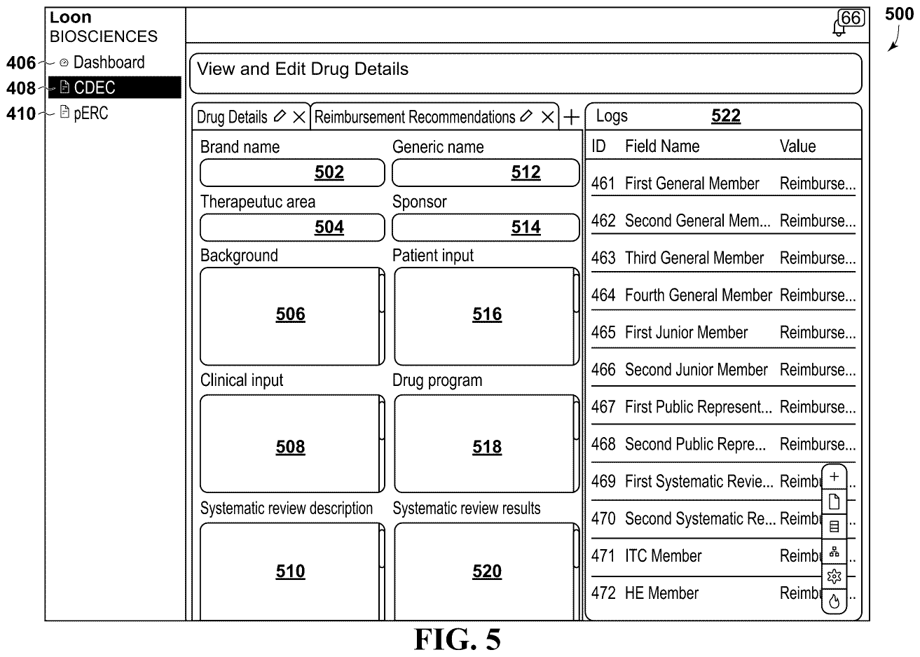

FIG. 5 illustrates a screenshot 500 of an interface for entering or managing drug information, displaying structured scenario data including “Brand name” 502, “Therapeutic area” 504, “Background” 506, “Clinical Input” 508, “Systematic Review Description” 510, “Generic Name” 512, “Sponsor” 514, “Patient Input” 516, “Drug Program” 518, “Systematic Review Results” 520, facilitating user review and scenario verification. Interface toggles include “Overview” 520 and “New” 520. The structured format of this interface assists users in inputting, reviewing, and validating comprehensive scenario information, for accuracy of scenario profiles utilized by the predictive modelling system for subsequent AI-driven deliberation simulations and predictive reimbursement analyses.

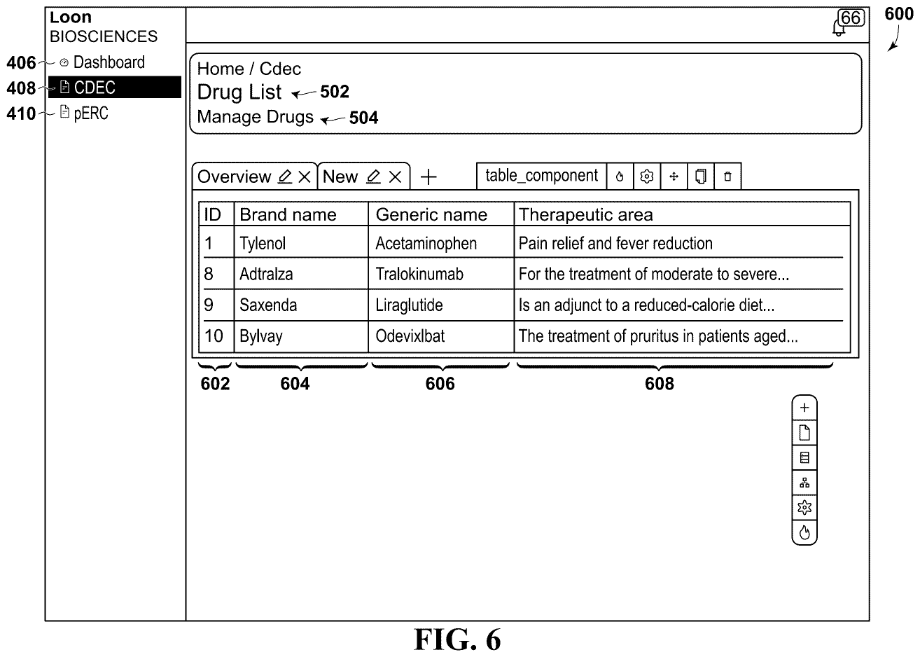

FIG. 6 illustrates a screenshot 600 of a drug list interface displaying structured drug information in table format, with columns for ID 602, Brand name 604, Generic name 606, and Therapeutic area 608, enabling selection and management of drug scenarios.

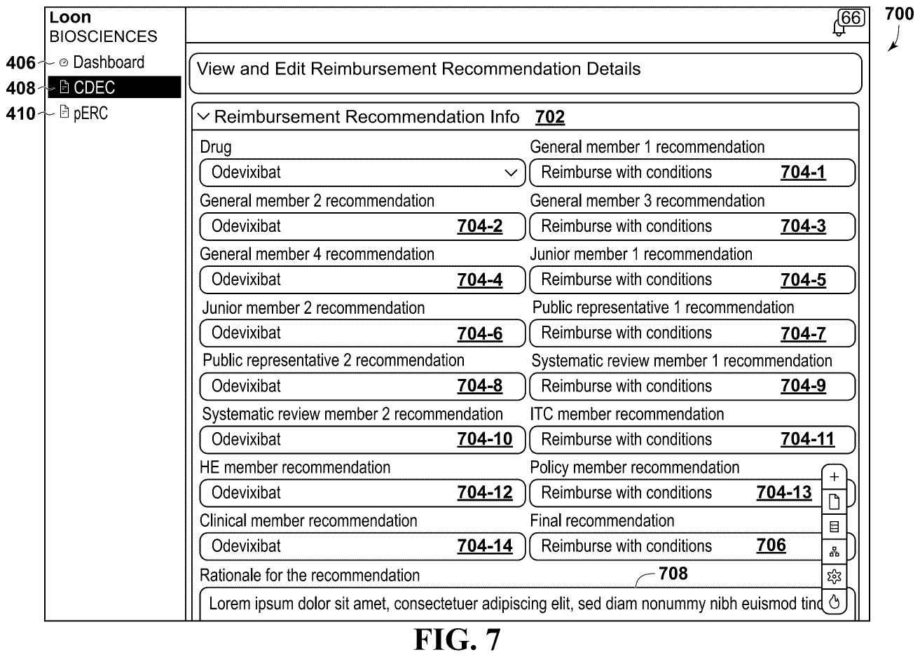

FIG. 7 illustrates a screenshot 700 of an interface summarizing simulated reimbursement decision outcomes. The “Reimbursement Recommendation Info” section 702 lists individual AI agents' recommendations 704-1, 704-2, . . . , 704-14. and presents the final synthesized recommendation 706 accompanied by a rationale 708. Screenshot 700 lists recommendations from general members (704-1, 704-2, 704-3, 704-4), junior members (704-5, 704-6), public representatives (704-7, 704-8), systematic review members (704-9, 704-10), indirect treatment comparison (ITC) member (704-11), health economics (HE) member (704-12), clinical member (704-13), and policy member (704-14). Each agent provides a recommendation, such as “Reimburse with conditions,” based on its fine-tuned domain expertise.

According to examples of the present specification, the clinical member 704-13 assesses the efficacy, safety, and unmet medical needs related to the drug, highlighting clinical trial results and potential clinical benefits and risks. The HE member 704-12 evaluates cost-effectiveness and budgetary impacts, considering economic models, affordability, and value-for-money analyses. The policy member 704-14 examines regulatory compliance, payer policies, and ethical considerations related to broader healthcare objectives. The systematic review members 704-9, 704-10 focus on robustness and quality of evidence, analyzing systematic reviews, evidence synthesis, and data consistency. The ITC member 704-11 specifically evaluates comparative efficacy and safety relative to existing alternative treatments. Public representatives 704-7, 704-8 emphasize patient perspectives, treatment burden, quality of life, and societal implications. General and junior members 704-1 to 704-6 synthesize diverse considerations, balancing clinical, economic, patient-related, and policy elements. The interface further provides a “Final recommendation” 706, which synthesizes individual agent evaluations into an overall reimbursement recommendation outcome, such as “Reimburse with conditions.” The accompanying detailed “Rationale for the recommendation” 708 explains how agent deliberations, clinical trial outcomes, statistical significance, clinically meaningful benefits, and potential limitations collectively informed the final recommendation. This structured representation facilitates transparent, actionable insights for stakeholders involved in reimbursement planning and strategic decision-making.

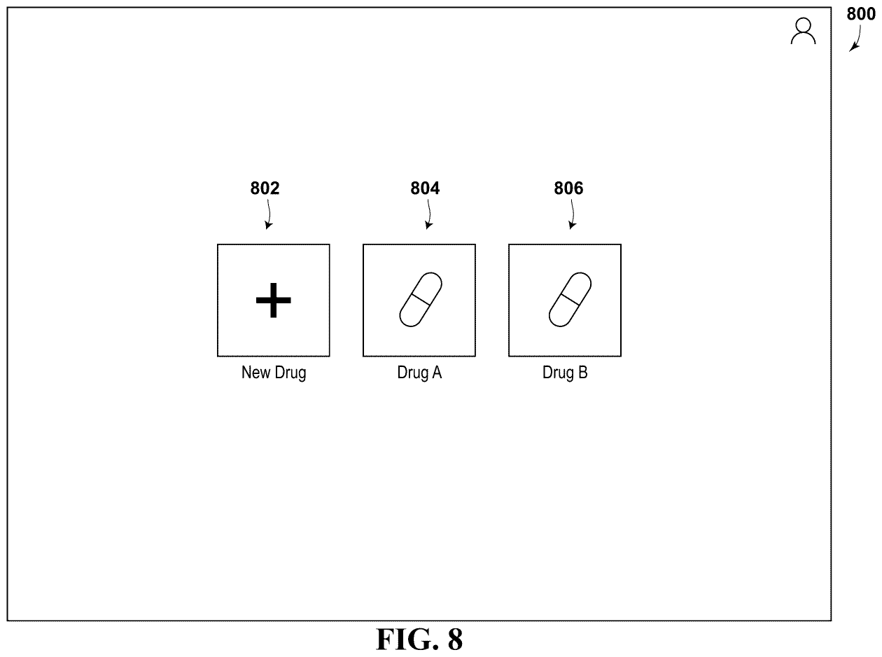

FIG. 8 through FIG. 11 illustrate various screenshots from a client application used with the HTA decision modelling system 100 according to examples of the present specification. FIG. 8 illustrates screenshot 800 with dashboard elements displaying initial user selection of a health technology scenario, including the selection or initiation of a new dossier. The dashboard includes a “+” or add button, which allows users to initiate a new health technology assessment scenario. Beside this, there is a display of buttons for existing dossier options, including “Drug A” 804 and “Drug B” 806, enabling users to select and modify previously created dossiers. The dashboard serves as the primary navigation point, allowing users to access dossier-related functionalities such as forecasting, evidence input, and strategic optimization.

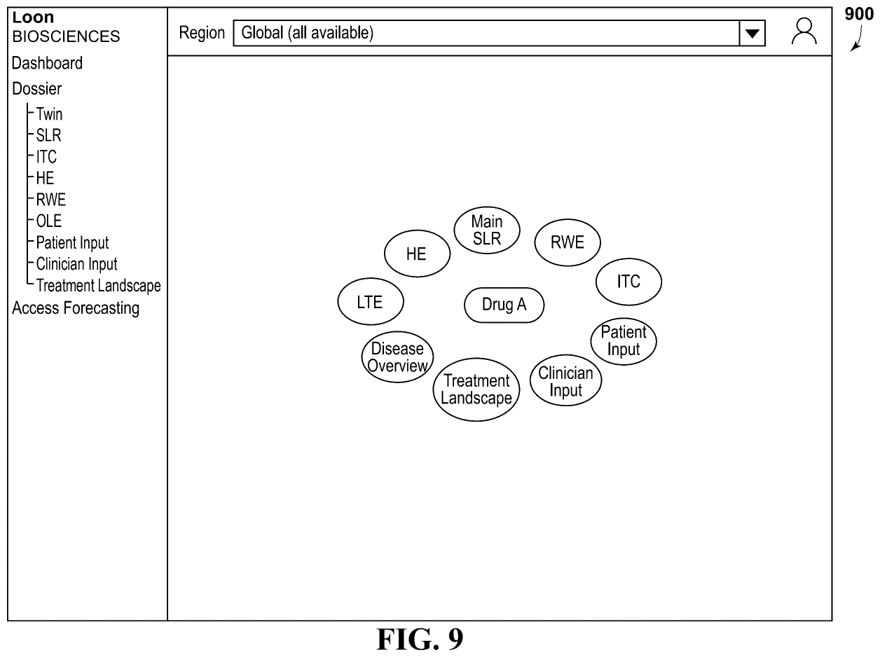

FIG. 9 illustrates screenshot 900 with a regional dashboard interface where an HTA dossier is selected or configured for a specific regional HTA assessment scenario. The dashboard navigation panel 902 includes options for accessing different sections: Dossier, Twin, Systematic Literature Review (SLR), Indirect Treatment Comparison (ITC), Health Economics (HE), Real-World Evidence (RWE), Overall Evidence (OLE), Patient Input, Clinician Input, Treatment Landscape, and Access Forecasting. The region selection module 904 allows users to choose the applicable regulatory body or reimbursement framework for their assessment. The committee deliberation visualization 906 depicts a drug assessment node, surrounded by multiple committee member elements arranged in a structured layout, resembling a roundtable discussion format. Each committee member node represents a distinct expert contributing to the deliberation process.

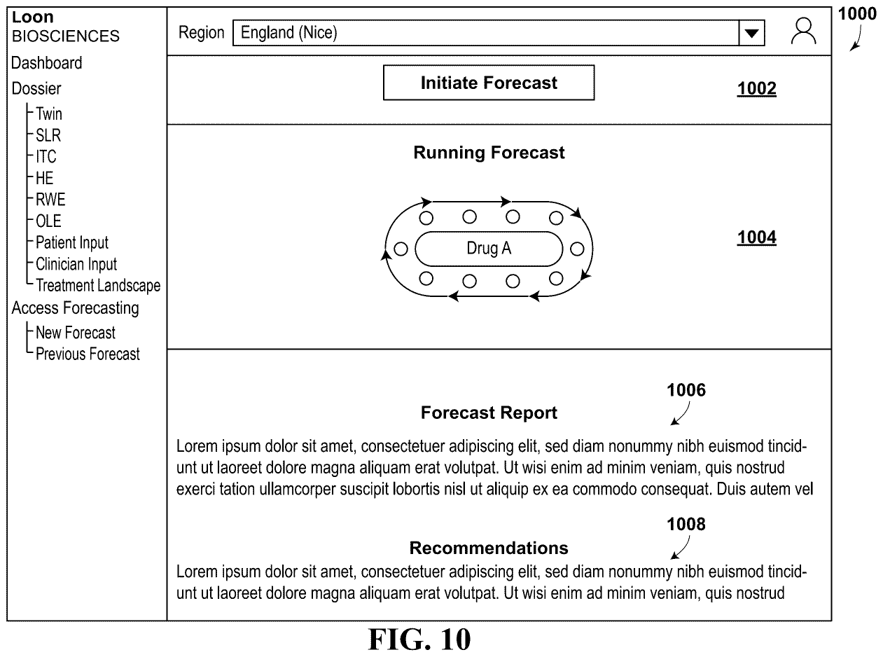

FIG. 10 illustrates screenshot 1000 with an interface for initiating and managing forecasting simulations, which is accessed by clicking the “Access Forecasting” button on the dashboard navigation panel 902. The forecast initiation button 1002 allows users to start a new forecast scenario for a selected dossier. The forecast progress panel 1004 displays active and previously completed forecast reports. The forecast report 1006 and recommendations panel 1008 provides AI-generated scenario-specific insights and optimization suggestions. FIG. 10 displays the simulated AI agent deliberation process visualization, showing multiple AI agents engaged in iterative deliberations around the submitted health technology, and demonstrating the intermediate simulation status.

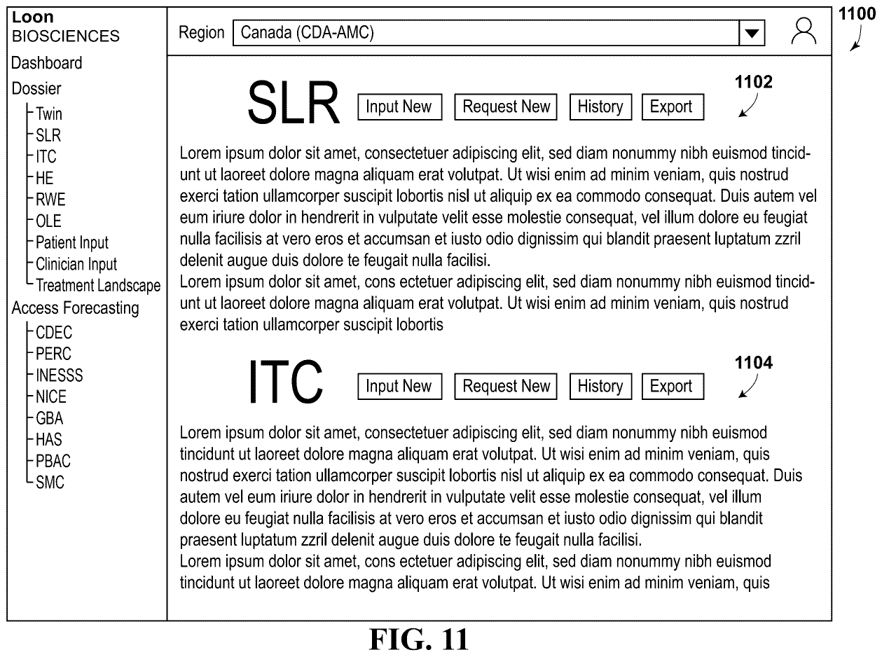

FIG. 11 illustrates screenshot 1100 with a dossier review and forecasting interface, which is accessed by clicking the “Dossier” button on the dashboard navigation panel 902. The interface includes an SLR management panel 1102, which provides options for handling systematic literature review data. This panel includes buttons labeled “SLR Input New” for entering new SLR data, “Request New” for initiating a new SLR request, “History” for accessing previous SLR entries, and “Export” for exporting SLR data. Similarly, the ITC management panel 1104 provides controls for managing indirect treatment comparison data, with buttons labeled “ITC Input New” for entering new ITC data, “Request New” for initiating a new ITC request, “History” for reviewing previous ITC analyses, and “Export” for exporting ITC data. This structured interface enables users to efficiently input, request, track, and export key evidence components required for the predictive HTA modelling workflow. Indirect Treatment Comparison (ITC) is a statistical method used to compare the efficacy or safety of different medical treatments when direct head-to-head clinical trials are unavailable. ITC allows researchers to evaluate multiple treatments by using a common comparator (e.g., placebo or standard therapy) to generate comparative effectiveness insights. This approach is widely used in HTA to support reimbursement decisions when direct comparative evidence is limited or non-existent.

With the generation of simulated reimbursement outcomes and actionable recommendations, the system 100 provides stakeholders with data-driven strategic insights, enabling transparent, and adaptable forecasting of reimbursement decisions, enhancing the effectiveness of HTA dossier submissions and reimbursement planning strategies.

One general aspect includes a method for predictive modelling of reimbursement decisions for a health technology, implemented by a computing system. The method includes the steps of: receiving, from an electronic user device, input data comprising a description of a health technology and parameters defining a target HTA scenario; analyzing the input data to generate a structured scenario profile for the health technology, using at least one generative AI model trained on historical HTA decision data; retrieving contextual data relevant to the structured scenario profile from a plurality of databases, wherein the databases comprise at least a historical HTA decision database, a clinical evidence database, and a health economic evidence database; simulating a deliberation process for evaluating the health technology in the target HTA scenario using a plurality of AI agents, wherein each of the AI agents is configured to represent a distinct expert perspective relevant to HTA deliberations, and wherein the AI agents interact iteratively by exchanging evaluations to determine a simulated reimbursement decision outcome; generating the simulated reimbursement decision outcome for the health technology based on the deliberation among the AI agents; generating one or more recommendations for modifying an HTA submission dossier associated with the health technology, wherein the recommendations are configured to increase a likelihood of receiving an affirmative reimbursement decision outcome; and providing, to the electronic user device, the simulated reimbursement decision outcome and the one or more recommendations.

Implementations may include one or more of the following features: employing a plurality of AI agents comprising at least a clinical evidence agent configured to evaluate clinical efficacy and safety data, a health economics agent configured to evaluate cost-effectiveness and budget impact data, and a policy expert agent configured to evaluate payer policy considerations and regulatory frameworks; conducting multiple iterative discussion cycles among the AI agents, wherein outputs generated by each of the AI agents during a given discussion cycle inform inputs provided to the AI agents in a subsequent discussion cycle, and wherein the iterative discussion cycles continue until a predetermined convergence criterion is satisfied; dynamically updating contextual data during the deliberation process to incorporate real-time data comprising at least one of newly published clinical trial results, updated clinical guidelines, or recent reimbursement decisions related to the health technology or comparator technologies; calculating a confidence score indicative of a probability associated with the simulated reimbursement decision outcome, wherein the confidence score is calculated based on a degree of consensus achieved among the AI agents or historical validation of similar simulations; identifying at least one modification to the HTA submission dossier from a set consisting of pricing adjustments or discount arrangements, additional clinical evidence generation, narrowed patient eligibility criteria, and risk-sharing or outcome-based reimbursement conditions, wherein the identified modification is determined based on factors predicted by the AI agents to increase the likelihood of an affirmative reimbursement decision outcome; implementing at least one of the plurality of AI agents as a large language model fine-tuned on historical transcripts and decision reports from past HTA committee deliberations; and retraining at least one of the plurality of AI agents using data from real-world HTA decision outcomes as feedback.

A system for predictive modelling of reimbursement decisions and health technology assessments is disclosed, including a memory storing instructions and one or more processors configured to execute the instructions. The instructions cause the processors to: receive, from an electronic user device, input data comprising a description of a health technology and parameters defining a target HTA scenario; generate a structured scenario profile for the health technology by analyzing the input data using a generative AI model trained on historical HTA decision data; retrieve, from a plurality of databases, contextual data relevant to the structured scenario profile, wherein the databases comprise a historical HTA decision database, a clinical evidence database, and a health economic evidence database; simulate an HTA committee deliberation process by instantiating a plurality of AI agents, wherein each of the AI agents represents a distinct HTA expert perspective, and wherein the AI agents iteratively exchange evaluations to simulate committee interactions; determine a simulated reimbursement decision outcome for the health technology based on evaluations exchanged by the plurality of AI agents; generate one or more recommendations for modifying an HTA submission dossier associated with the health technology, wherein the recommendations are configured to increase a likelihood of receiving an affirmative reimbursement decision outcome; and output, for display on the electronic user device, the simulated reimbursement decision outcome and the one or more recommendations.

A non-transitory computer-readable storage medium is disclosed. The storage medium stores instructions that, when executed by at least one processor, cause the at least one processor to perform operations comprising: receiving, via a user interface, input data defining a health technology and parameters characterizing a target HTA scenario; converting the input data into a structured scenario representation using an AI technique; retrieving relevant contextual data for the structured scenario representation from a plurality of databases, including historical HTA decision data, clinical evidence, and economic evidence; simulating an HTA committee deliberation using multiple AI agents, each AI agent representing a distinct expert viewpoint relevant to HTA deliberations, wherein the AI agents iteratively exchange evaluations to deliberate on a reimbursement decision outcome; generating a simulated reimbursement decision outcome for the health technology based on deliberations among the AI agents; and providing, via the user interface, the simulated reimbursement decision outcome along with one or more recommended modifications to the HTA submission dossier, wherein the recommended modifications are configured to increase the likelihood of an affirmative reimbursement decision outcome. Implementations may further include employing AI agents based on large language models, wherein the AI agents communicate in natural language and iteratively update their evaluations based on arguments presented by other AI agents during the simulated deliberation, and generating a report highlighting specific factors identified by the AI agents as negatively impacting the reimbursement decision outcome, wherein the report includes targeted suggestions for addressing each identified factor in a revised HTA submission dossier or reimbursement negotiation strategy.

While the invention has been described with reference to the specific embodiments, it will be understood by those skilled in the art that various changes may be made without departing from the scope of the present specification. Furthermore, the scope of the present specification is not intended to be limited to the specific embodiments described herein. Additionally, the range of embodiments described herein is not intended to limit the scope of the present specification. Rather, the invention encompasses all modifications and variations within the scope of the present specification.

1. A method for predictive modelling of reimbursement decisions for a health technology, implemented by a computing system, comprising:
receiving, from an electronic user device, input data comprising a description of a health technology and parameters defining a target Health Technology Assessment (HTA) scenario;
analyzing the input data to generate a structured scenario profile for the health technology, using at least one generative artificial intelligence (AI) model trained on historical HTA decision data;
retrieving contextual data relevant to the structured scenario profile from a plurality of databases, wherein the databases comprise at least a historical HTA decision database, a clinical evidence database, and a health economic evidence database;
simulating a deliberation process for evaluating the health technology in the target HTA scenario using a plurality of AI agents, wherein each of the AI agents is configured to represent a distinct expert perspective relevant to HTA deliberations, and wherein the AI agents interact iteratively by exchanging evaluations to determine a simulated reimbursement decision outcome;
generating the simulated reimbursement decision outcome for the health technology based on the deliberation among the AI agents;
generating one or more recommendations for modifying an HTA submission dossier associated with the health technology, wherein the recommendations are configured to increase a likelihood of receiving an affirmative reimbursement decision outcome; and
providing, to the electronic user device, the simulated reimbursement decision outcome and the one or more recommendations.

2. The method of claim 1, wherein the plurality of AI agents comprises at least:
a clinical evidence agent configured to evaluate clinical efficacy and safety data;
a health economics agent configured to evaluate cost-effectiveness and budget impact data; and
a policy expert agent configured to evaluate payer policy considerations and regulatory frameworks.

3. The method of claim 1, wherein simulating the deliberation process further comprises conducting a plurality of iterative discussion cycles among the AI agents, wherein outputs generated by each of the AI agents during a given discussion cycle inform inputs provided to the AI agents in a subsequent discussion cycle, and wherein the iterative discussion cycles continue until a predetermined convergence criterion is satisfied.

4. The method of claim 1, further comprising dynamically updating the contextual data during the deliberation process to incorporate real-time data comprising at least one of newly published clinical trial results, updated clinical guidelines, or recent reimbursement decisions related to the health technology or comparator technologies.

5. The method of claim 1, further comprising calculating a confidence score indicative of a probability associated with the simulated reimbursement decision outcome, wherein the confidence score is calculated based on a degree of consensus achieved among the AI agents or historical validation of similar simulations.

6. The method of claim 1, wherein generating the one or more recommendations comprises identifying at least one modification to the HTA submission dossier from a set consisting of:
pricing adjustments or discount arrangements;
additional clinical evidence generation;
narrowed patient eligibility criteria; and
risk-sharing or outcome-based reimbursement conditions, wherein the identified modification is determined based on factors predicted by the AI agents to increase the likelihood of an affirmative reimbursement decision outcome.

7. The method of claim 1, wherein at least one of the plurality of AI agents comprises a large language model fine-tuned on historical transcripts and decision reports from past HTA committee deliberations.

8. The method of claim 1, further comprising retraining at least one of the plurality of AI agents using data from real-world HTA decision outcomes as feedback.

9. A system for predictive modelling of reimbursement decisions and health technology assessments, comprising:
a memory storing instructions; and
one or more processors configured to execute the instructions to perform operations comprising:
receiving, from an electronic user device, input data comprising a description of a health technology and parameters defining a target Health Technology Assessment (HTA) scenario;
generating a structured scenario profile for the health technology by analyzing the input data using a generative artificial intelligence (AI) model trained on historical HTA decision data;
retrieving, from a plurality of databases, contextual data relevant to the structured scenario profile, wherein the databases comprise a historical HTA decision database, a clinical evidence database, and a health economic evidence database;
simulating an HTA committee deliberation process by instantiating a plurality of AI agents, wherein each of the AI agents represents a distinct HTA expert perspective, and wherein the AI agents iteratively exchange evaluations to simulate committee interactions;
determining a simulated reimbursement decision outcome for the health technology based on evaluations exchanged by the plurality of AI agents;
generating one or more recommendations for modifying an HTA submission dossier associated with the health technology, wherein the recommendations are configured to increase a likelihood of receiving an affirmative reimbursement decision outcome; and
outputting, for display on the electronic user device, the simulated reimbursement decision outcome and the one or more recommendations.

10. The system of claim 9, wherein the plurality of AI agents comprises:
a clinical evidence agent specialized in evaluating clinical trial data;
a health economics agent specialized in evaluating economic modelling data; and
a policy expert agent specialized in evaluating payer and regulatory policy considerations;
and wherein the one or more processors are further configured to execute a deliberation orchestration module to manage the iterative interaction among the AI agents to emulate an HTA committee deliberation.

11. The system of claim 9, wherein the one or more processors are further configured to execute an outcome prediction engine that aggregates evaluations provided by the plurality of AI agents to generate the simulated reimbursement decision outcome accompanied by a synthesized rationale reflecting consensus or divergence among the AI agents.

12. The system of claim 9, wherein the plurality of databases comprises a database of historical health technology assessment outcomes, and wherein the one or more processors are further configured to execute a training module that periodically updates the generative AI model and at least one of the plurality of AI agents using data from the historical health technology assessment outcomes database.

13. At least one non-transitory computer-readable storage medium storing instructions that, when executed by at least one processor, cause the at least one processor to perform operations comprising:
receiving, via a user interface, input data defining a health technology and parameters characterizing a target health technology assessment (HTA) scenario;
converting the input data into a structured scenario representation using an artificial intelligence (AI) technique;
retrieving relevant contextual data for the structured scenario representation from a plurality of databases, including historical HTA decision data, clinical evidence, and economic evidence;
simulating an HTA committee deliberation using multiple AI agents, each AI agent representing a distinct expert viewpoint relevant to HTA deliberations, wherein the AI agents iteratively exchange evaluations to deliberate on a reimbursement decision outcome;
generating a simulated reimbursement decision outcome for the health technology based on deliberations among the AI agents; and
providing, via the user interface, the simulated reimbursement decision outcome along with one or more recommended modifications to the HTA submission dossier, wherein the recommended modifications are configured to increase the likelihood of an affirmative reimbursement decision outcome.

14. The non-transitory computer-readable storage medium of claim 13, wherein the instructions for simulating the HTA committee deliberation further comprise instructions for employing AI agents based on large language models, the AI agents configured to communicate with one another in natural language, iteratively updating their evaluations based on arguments presented by other AI agents during the simulated deliberation.

15. The non-transitory computer-readable storage medium of claim 13, wherein providing the one or more recommended modifications further comprises generating a report highlighting specific factors identified by the AI agents as negatively impacting the reimbursement decision outcome and providing targeted suggestions for addressing each identified factor in a revised HTA submission dossier or reimbursement negotiation strategy.
